(19) United States
(12) Patent Application Publication
(10) Pub. No.: US 2024/0043447 A1
(43) Pub. Date: Feb. 8, 2024

(54) SYNTHESIS OF HYDROMORPHONE BASE

(71) Applicant: Ferrer Internacional, S.A., Sant Cugat del Vallès (Barcelona) (ES)

(72) Inventors: Francisco De Asís MARQUILLAS OLONDRIZ, SANT CUGAT DEL VALLÈS (ES); Jorge BESSA BELLMUNT, BELLATERRA (ES); Antonio Abelino DE LEÓN MARTÍN, BELLATERRA (ES)

(73) Assignee: Ferrer Internacional, S.A., Sant Cugat del Vallès (Barcelona) (ES)

(21) Appl. No.: 18/036,126

(22) PCT Filed: Nov. 12, 2021
(86) PCT No.: PCT/EP2021/081515

§ 371 Date: May 9, 2023

(30) Foreign Application Priority Data
Nov. 13, 2020 (EP) 20382987.4
Dec. 16, 2020 (EP) 20383101.1
Jul. 23, 2021 (EP) 21187433.4

(51) Int. Cl. C07D 498/08

(52) U.S. Cl. CPC C07D 498/08

(57) ABSTRACT

Hydromorphone hydrochloride (I·HCl) is converted into hydromorphone base (I) via hydromorphone monohydrate (I·H₂O).

### FIELD OF THE INVENTION

This patent belongs to the field of preparation and purification of hydromorphone base (I).

### BACKGROUND OF THE INVENTION

Hydromorphone is a potent opioid used to treat severe pain. Hydromorphone and some related species have the following chemical structures:

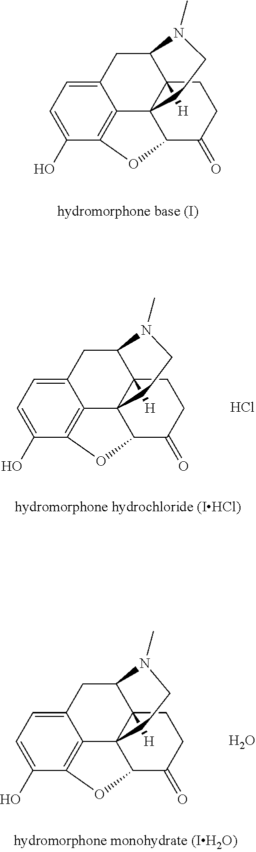

Hydromorphone is also known as dihydromorphinone. Hydromorphone hydrochloride (MCI) is marketed under several trademarks such as Dilaudid®, Exalgo® or Palladone® among other Trade Names.

The most common form of hydromorphone is the hydrochloride (MCI). One part of hydromorphone hydrochloride (MCI) dissolves in three parts of water. A low water solubility form of hydromorphone such as hydromorphone base (I) is useful for some applications.

The inventors have found that the acid/base equilibrium of hydromorphone is complex and that it was not straightforward to obtain hydromorphone base (I) form hydromorphone hydrochloride (MCI). According to the MERK INDEX, 2006 hydromorphone has one acidic and one basic point with pKa=8.92 and pKb 7.0:

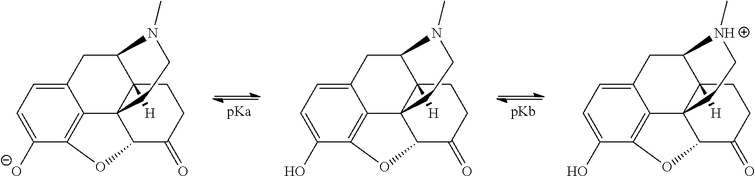

Using the hydromorphone monohydrate (I·H₂O) as an intermediate, hydromorphone base (I) can be obtained from hydromorphone hydrochloride (MCI) easily and with good yields and purity.

In the prior art, solid hydromorphone base (I) has been prepared from the following hydromorphone hydrochloride (MCI) solutions:
from water (GOMEZ, et al., 2014, WO15011474 A1, US2015252052 AA (pH 7.5), WO06005112 A1 (pH 9.1, KOH adjusted), WO11137086 A1 (pH 9.0), WANG, et al., 2020 (pH adjusted with NH₄OH), U.S. Pat. No. 2,628,962 A (NH₄OH adjusted), U.S. Pat. No. 2,654,756 A (NH₄OH adjusted, m.p. 262.5-263° C.), or WO0134608 A1 (pH 8.8, NH₄OH adjusted)),
from water/ethanol solution (WO05100361 A1 (pH 10-10.5)),
from water/1-butanol mixture (WO06005112 A1 (4:1, pH 9.1 (NaOH adjusted)),
from water/2-propanol solution (WO10118271 A1 (pH 8.7-9.1, NH₄OH adjusted)),
from methanol solutions (CSUK, et al., 2012 (m.p. 264-266° C.), WO05047291 A1, WO05113557 A1, WO06104656 A1, U.S. Pat. No. 7,399,859 BA, US2015252052 AA or WO18009856 A1),
recrystallization in ethanol (WO06005112 A1 (m.p. 264-266° C., FTIR (KBr): 1729 (C═O) cm⁻¹), RAPOPORT, et al., 1950 (m.p. 266-267° C. (either by removal of solvent from a ethyl acetate solution or recrystallization in ethanol), WO9805667 A1, WO15134003 A1, or US2015252052 AA),
from acetone/2-propanol mixture (ERBING, et al., 2016),
from CH₂Cl₂:MeOH mixture (MURPHY, et al., 2014 (m.p.>230° C.), or US2015225419 AA (6:1)),
recrystallized from CH₂Cl₂/heptane (MURPHY, et al., 2014 (m.p.>230° C.)),
from ethyl acetate/MeOH solution (WO9805667 A1),
from ethyl acetate (U.S. Pat. No. 2,649,454 A),
from acetonitrile/ethanol/water solution (WO11137086 A1 (pH 8.7-9.2, NH₄OH adjusted)),
from THF/acetone solution (MAZUREK, et al., 2016, m.p. 276.7° C. (548.8 K, referred as Form I)), or
from ethanol/toluene (MAZUREK, et al., 2016, m.p. 277.0° C. (550.2 K, referred as Form II)).

According to FISCHER, et al., 1949, hydromorphone base (I) melts with decomposition at 257° C.

In WO06091885 A2 D1-hydromorphone, D2-hydromorphone, and D3-hydromorphone monohydrates are precipitated from a CHCl₃/aqueous NH₄OH mixture.

In CN108164540 A, a recrystallization process is mentioned, but the solvent is not specified.

### SUMMARY OF THE INVENTION

One aspect of the present invention is a process for preparing hydromorphone base (I) comprising:
i. suspending or dissolving hydromorphone monohydrate (I·H₂O) in at least an organic solvent, and
ii. isolating hydromorphone base (I).

Another aspect of the present invention is hydromorphone monohydrate (I·H₂O)

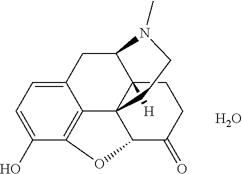

Another aspect of the present invention is a process for preparing hydromorphone monohydrate (I·H₂O) comprising
a) dissolving a hydromorphone salt in a solvent medium comprising water,
b) adjusting the pH of the mixture of step a) between 8 and 10, and
c) isolating hydromorphone monohydrate (I·H₂O) from the mixture of step b)
wherein
in step b) the pH is adjusted with a weak base.

### Definitions

Within the present document, the following terms are used with the following meanings.

“Solvent” refers to a liquid that serves for the medium of a reaction.

“Organic solvent” refers to a solvent mainly comprising organic compounds.

“Polar organic solvent” refers to an organic solvent wherein its molecules have large dipoles, i.e. includes bonds between atoms of different electronegativity. A solvent is considered to be polar when it has a relative permittivity (formerly known as dielectric constant) higher than 15.

“Relative permittivity” is the ratio of the electric field strength in vacuum to that in a given medium. It was formerly called the dielectric constant. Relative permittivity is, thus, a dimensionless figure.

“Polar protic organic solvent” refers to an organic polar solvent wherein its molecules have O—H or N—H bonds.

“Polar aprotic organic solvent” refers to an organic polar solvent wherein its molecules lack O—H or N—H bonds.

“Non-polar organic solvent” refers to an organic solvent wherein its molecules do not contain bonds between atoms of different electronegativity. A solvent is considered to be non-polar when it has a relative permittivity (formerly known as dielectric constant) lower than 15.

“Isotopically labelled” refers to a non-radioactive substance wherein one or more of its atoms have been enriched with a stable isotope that is not the naturally occurring most abundant isotope. Suitable stable isotopes to enrich include isotopes of hydrogen, such as ²H (usually referred as deuterium, D) and ³H (usually referred as tritium, T); carbon, such as ¹¹C, ¹³C and ¹⁴C; nitrogen, such as ¹³N and ¹⁵N; oxygen, such as ¹⁵O, ¹⁷O and ¹⁸O.

“Weak base” is a base that it is not fully dissociated when dissolved in water.

“Strong base” is a base that is fully dissociated when dissolved in water.

“FTIR” refers to Fourier-Transform Infra-Red spectroscopy.

“PXRD” refers to Powder X-Ray Diffraction.

“DSC” refers to Differential Scanning calorimetry.

“TGA” refers to Thermo Gravimetric Analysis.

“Pharmaceutically acceptable excipient” refers to any substance, other than the pharmacologically active drug or prodrug, that is useful in preparing a pharmaceutical composition, which is generally safe and non-toxic and that is approved or approvable by a regulatory agency.

### DESCRIPTION OF FIGURES

FIG. 1: FTIR of hydromorphone monohydrate (I·H₂O) Form A prepared in Example 1.

FIG. 2: DSC of hydromorphone monohydrate (I·H₂O) Form A prepared in Example 1 showing an endotherm peak at 112° C. due to the loss of water and at 276° C. due to the melting of the solid.

FIG. 3: TGA of hydromorphone monohydrate (I·H₂O) Form A prepared in Example 1 showing a 5.8% drop between 98° C. and 115° C. due to the loss of water. One molecule of water represents a theoretical 5.94% of the molecular weight.

FIG. 4: FTIR of hydromorphone base (I) prepared in Example 2.

FIG. 5: DSC of hydromorphone base (I) prepared in Example 2 showing an endotherm peak 275° C.

FIG. 6: TGA of hydromorphone base (I) prepared in Example 2 showing no loss of water.

FIG. 7: PXRD of hydromorphone monohydrate (I·H₂O) Form A prepared in Example 1.

FIG. 8: FTIR of hydromorphone hydrochloride (I·HCl) with the following picks 3029, 2960, 2926, 2584, 1716, 1638, 1621, 1312, 976, 736.

FIG. 9: FTIR of hydromorphone hydrochloride (MCI), hydromorphone base (I), and hydromorphone monohydrate (I·H₂O) Form A superposed.

### DETAILED DESCRIPTION OF THE INVENTION

The processes are schematically represented in the following scheme:

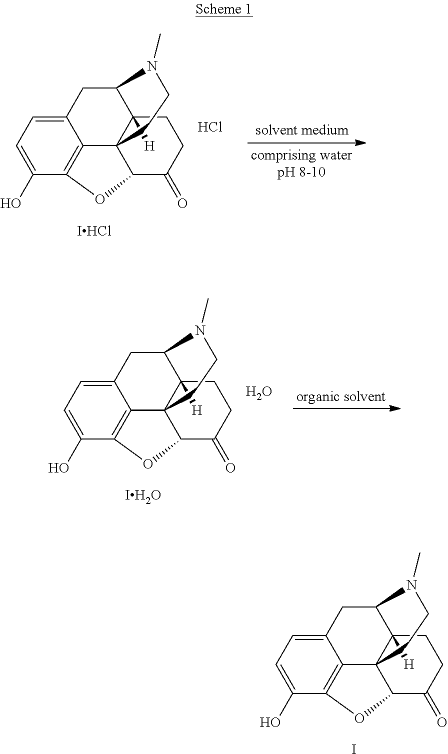

DSC and TGA experiments are performed between 30 and 350° C. at 10° C./min under N₂ flux.

Embodiment 1. A process for preparing hydromorphone base (I) comprising:
i. suspending or dissolving hydromorphone monohydrate (I·H₂O) in at least an organic solvent, and
ii. isolating hydromorphone base (I).

Embodiment 2. The process of the previous embodiment, wherein the organic solvent comprises primary, secondary or tertiary alcohols of C₁ to C₈ linear or branched alkanes; C₁ to C₈ linear or branched alkyl esters of C₁ to C₅ linear or branched carboxylic acids; ethers with the same or two different C₁ to C₆-linear or branched chains attached to the oxygen in cyclic or open forms; C₁ to C₈ linear or branched alkanes substituted by 1 to 4 different or same halogen, wherein the halogen is selected from Cl, Br or I; ketones of same or different C₁ to C₈ linear or branched alkanes chains; C₁ to C₅ nitriles; C₅ to C₈ linear, branched or cyclic alkanes; C₁ to C₃ mono, di or tri alkyl substituted C₅ to C₈ aromatic or heteroaromatic compounds; or mixtures thereof.

Embodiment 3. The process of any of the previous embodiments, wherein the organic solvent comprises methanol, ethanol, 1-propanol, 2-propanol, 1-butanol, 2-butanol, isobutanol, tert-butanol, methyl acetate, ethyl acetate, isopropyl acetate, tetrahydrofuran, dichloromethane, acetone, methyl isobutyl ketone, acetonitrile, propionitrile, butyronitrile, diethyl ether, 1,4-dioxane, methyl tert-butyl ether, pentane, cyclopentane, hexane, cyclohexane, heptane, toluene, chloroform, or mixtures thereof.

Embodiment 4. The process of any of the previous embodiments, wherein the organic solvent comprises ethyl acetate, isopropyl acetate, tetrahydrofuran, methyl isobutyl ketone, dichloromethane, methanol, ethanol, propanol, isopropanol, 1-butanol, isobutanol, tert-butanol, pentane, hexane, heptane, toluene, methyl tert-butyl ether, diethyl ether, or mixtures thereof.

Embodiment 5. The process of any of the previous embodiments, wherein the organic solvent comprises ethyl acetate, isopropyl acetate, tetrahydrofuran, methyl isobutyl ketone, methanol, heptane, toluene, methyl tert-butyl ether, or mixtures thereof.

Embodiment 6. The process of any of the previous embodiments, wherein the organic solvent comprises ethyl acetate, isopropyl acetate, methyl isobutyl ketone, heptane, toluene, methyl tert-butyl ether, or mixtures thereof.

Embodiment 7. The process of any of the previous embodiments, wherein the organic solvent comprises a polar organic solvent.

Embodiment 8. The process of the previous embodiment, wherein the polar organic solvent comprises methanol, ethanol, 1-propanol, 2-propanol, 1-butanol, 2-butanol, isobutanol, tert-butanol, methyl acetate, ethyl acetate, isopropyl acetate, tetrahydrofuran, dichloromethane, acetone, methyl isobutyl ketone, acetonitrile, propionitrile, butanenitrile, or mixtures thereof.

Embodiment 9. The process of the previous embodiment, wherein the organic solvent comprises a polar protic organic solvent.

Embodiment 10. The process of the previous embodiment, wherein the polar protic organic solvent comprises methanol, ethanol, 1-propanol, 2-propanol, 1-butanol, 2-butanol, isobutanol, tert butanol, or mixtures thereof.

Embodiment 11. The process of embodiment 8, wherein the organic solvent comprises a polar aprotic organic solvent.

Embodiment 12. The process of the previous embodiment, wherein the polar aprotic organic solvent comprises methyl acetate, ethyl acetate, isopropyl acetate, tetrahydrofuran, dichloromethane, acetone, methyl isobutyl ketone, acetonitrile, propionitrile, butanenitrile, or mixtures thereof.

Embodiment 13. The process of any of the embodiments 1 to 6, wherein the organic solvent comprises a non-polar organic solvent.

Embodiment 14. The process of the previous embodiment, wherein the non-polar organic solvent comprises diethyl ether, 1,4-dioxane, methyl tert-butyl ether, pentane, cyclopentane, hexane, cyclohexane, heptane, toluene, chloroform, or mixtures thereof.

Embodiment 15. The process of any of the previous embodiments, wherein the organic solvent comprises less than 5% of water.

Embodiment 16. The process of the previous embodiment, wherein the organic solvent comprises less than 3% of water.

Embodiment 17. The process of the previous embodiment, wherein the organic solvent comprises less than 2% of water.

Embodiment 18. The process of the previous embodiment, wherein the organic solvent comprises less than 1% of water.

Embodiment 19. The process of the previous embodiment, wherein the organic solvent comprises less than 0.1% of water.

Embodiment 20. The process of the previous embodiment, wherein the organic solvent comprises less than % of water.

Embodiment 21. The process of the any of the previous embodiments, wherein the organic solvent is an anhydrous organic solvent.

Embodiment 22. The process of any of the previous embodiments, wherein the hydromorphone monohydrate (I·H₂O) is suspended or dissolved in step i. at 15-80° C.

Embodiment 23. The process of the previous embodiment, wherein the hydromorphone monohydrate (I·H₂O) is suspended or dissolved in step i. at 25-50° C.

Embodiment 24. The process of the previous embodiment, wherein the hydromorphone monohydrate (I·H₂O) is suspended or dissolved in step i. at 25-35° C.

Embodiment 25. The process of any of the embodiments 1 to 22, wherein the hydromorphone monohydrate (I·H₂O) is suspended or dissolved in step i. at 20-25° C.

Embodiment 26. The process of the previous embodiment, wherein the hydromorphone monohydrate (I·H₂O) is suspended or dissolved in step i. at room temperature.

Embodiment 27. The process of any of the previous embodiments, wherein isolating step ii. is performed between −5 and 25° C.

Embodiment 28. The process of any of the previous embodiments, wherein the isolating step ii. is performed between −5 and 15° C.

Embodiment 29. The process of any of the previous embodiments, wherein the isolating step ii. is performed between 0 and 10° C.

Embodiment 30. The process of any of the embodiments 1 to 29 or 76 to 88, wherein the hydromorphone monohydrate (I·H₂O) is prepared according to embodiments 47 to 67.

Embodiment 31. The process of any of the embodiments 1 to 29 or 76 to 88, wherein the hydromorphone monohydrate (I·H₂O) is the hydromorphone monohydrate (I·H₂O) as described in embodiments 36 to 43 or 68 to 75.

Embodiment 32. A crystalline form of hydromorphone base (I) having a DSC endothermic peak at 275±2° C.

Embodiment 33. The crystalline form of hydromorphone base (I) of the previous embodiment characterized by an FTIR comprising the following peaks 3361, 2924, 2797, 1727, 1502, 1314, 946±5 cm⁻¹.

Embodiment 34. A pharmaceutical composition comprising a crystalline form of hydromorphone base (I) as described in any of the embodiments 32 to 33, together with at least one pharmaceutically acceptable excipient.

Embodiment 35. A crystalline form of hydromorphone base (I) as described in any of the embodiments 32 to 33 or the pharmaceutical composition as described in embodiment 34 for use in the treatment of pain.

Embodiment 36. Hydromorphone monohydrate (I·H₂O).

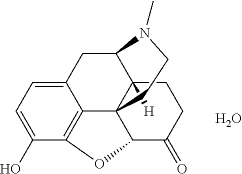

Embodiment 37. The hydromorphone monohydrate (I·H₂O) of the previous embodiment which is to be considered that does not encompass the isotopically labelled derivatives.

Embodiment 38. The hydromorphone monohydrate (I·H₂O) of the previous embodiment, wherein the non-majoritarian isotopes represent less than 5% in mole percent.

Embodiment 39. The hydromorphone monohydrate (I·H₂O) of the previous embodiment, wherein the less abundant isotopes represent less than 3% in mole percent.

Embodiment 40. The hydromorphone monohydrate (I·H₂O) of any of the embodiments 36 to 39, wherein deuterium represents less than 0.1% of the hydrogen isotopes in mole percent.

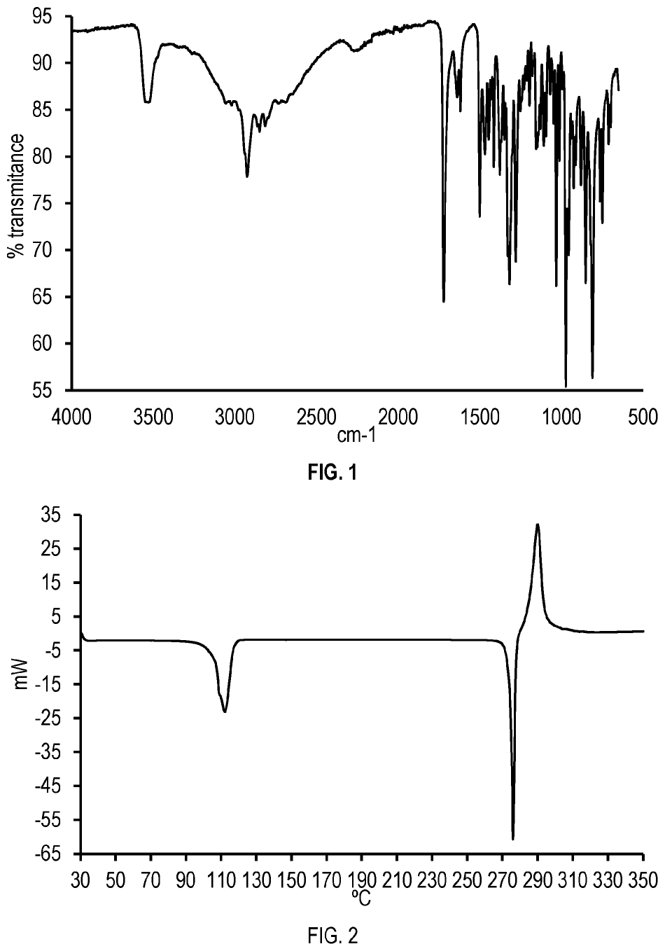

Embodiment 41. A hydromorphone monohydrate (I·H₂O) of any of the embodiments 36 to 40 in a crystalline form characterized by an FTIR substantially such as that in FIG. 1.

Embodiment 42. The hydromorphone monohydrate (I·H₂O) crystalline Form A of any of the embodiments 36 to 41 characterized by an FTIR comprising the following peaks: 3547, 2925, 1721, 1377, 973, 749±5 cm⁻¹.

Embodiment 43. The hydromorphone monohydrate (I·H₂O) crystalline Form A of the previous embodiment characterized by a DSC with two endotherms peaks, one at 112±4° C. and a second at 276±2° C.

Embodiment 44. Hydromorphone monohydrate (I·H₂O), as described in embodiments 36 to 43, for use in the preparation of hydromorphone base (I).

Embodiment 45. A pharmaceutical composition comprising a hydromorphone monohydrate (I·H₂O) as described in any of the embodiments 36 to 43, together with at least one pharmaceutically acceptable excipient.

Embodiment 46. A hydromorphone monohydrate (I·H₂O) as described in any of the embodiments 36 to 43 or the pharmaceutical composition as described in embodiment 45 for use in the treatment of pain.

Embodiment 47. A process for preparing hydromorphone monohydrate (I·H₂O) comprising
a) dissolving a hydromorphone salt in a solvent medium comprising water,
b) adjusting the pH of the mixture of step a) between 8 and 10, and
c) isolating hydromorphone monohydrate (I·H₂O) from the mixture of step b).

Embodiment 48. The process of the previous embodiment, wherein the hydromorphone salt is hydromorphone hydrochloride (I·HCl), sulphate, 1,4-benzendicarboxylate, 1,1,1-trifluoromethansulfonate, 1-hydroxy-2-naphthalenecarboxylate, 3-hydroxy-2-naphthalenecarboxylate, α-methyl-4-[(2-oxocyclopentyl)methyl]benzeneacetate, 2′,4′-difluoro-4-hydroxy[1,1,1-biphenyl]-3-carboxylate, 4,5-diphenyl-2-oxazolepropanoate, α-methyl-3-phenoxybenzeneacetate, 1-methyl-5-(4-methylbenzoyl)-1H-pyrrole-2-acetate, 2-fluoro-α-methyl[1,1′-biphenyl]-4-acetate, α-methyl-4-(2-thienylcarbonyl)benzeneacetate, (1Z)-5-fluoro-2-methyl-1-[[4-(methylsulfinyl)phenyl]methylene]-1H-indene-3-acetate, 3-benzoyl-α-methylbenzeneacetate, 1,8-diethyl-1,3,4,9-tetrahydropyrano[3,4-b]indole-1-acetate, (αS)-6-methoxy-α-methyl-2-naphthaleneacetate, 2-naphthalenesulfonate, butanedioate, (2E)-2-butenedioate, (2Z)-2-butenedioate, 2-hydroxy-1,2,3-propanetricarboxylate, 4-methylbenzenesulfonate, 2-hydroxypropanoate, benzoate, borate, dodecanoate, ethanedioate, pentanoate, hydrobromide, (2R,3R)-2,3-dihydroxybutanedioate, phosphate, acetate, (9Z)-9-octadecenoate, 2,3-dimethyl-4-[[(4-methylphenyl)sulfonyl]oxy]benzenesulfonate, 4-[[(2,3-dimethylphenyl)sulfonyl]oxy]benzenesulfonate, 4-[[(4-methylphenyl)sulfonyl]oxy]benzenesulfonate, 4-[(phenylsulfonyl)oxy]benzenesulfonate, or 3-hydroxy-2,6-dimethylbenzenesulfonate.

Embodiment 49. The process of the previous embodiment, wherein the hydromorphone salt is hydromorphone hydrochloride (I·HCl).

Embodiment 50. The process of embodiment any of the embodiments 47 to 49, wherein the solvent medium comprising water comprises at least 50% water.

Embodiment 51. The process of the previous embodiment, wherein the solvent medium comprising water comprises at least 75% water.

Embodiment 52. The process of the previous embodiment, wherein the solvent medium comprising water comprises at least 85% water.

Embodiment 53. The process of the previous embodiment, wherein the solvent medium comprising water comprises at least 95% water.

Embodiment 54. The process of embodiment any of the embodiments 47 to 53, wherein the pH is adjusted between 8.2 and 9.8.

Embodiment 55. The process of the previous embodiment, wherein the pH is adjusted between 8.4 and 9.6.

Embodiment 56. The process of the previous embodiment, wherein the pH is adjusted between 8.6 and 9.4.

Embodiment 57. The process of the previous embodiment, wherein the pH is adjusted between 8.8 and 9.2.

Embodiment 58. The process of any of the embodiments 47 to 57, wherein step c) is performed between −5 and 25° C.

Embodiment 59. The process of the previous embodiment, wherein step c) is performed between −5 and 15° C.

Embodiment 60. The process of the previous embodiment, wherein step c) is performed between −2 and 12° C.

Embodiment 61. The process of the previous embodiment, wherein step c) is performed between 0 and 10° C.

Embodiment 62. The process of any of the embodiments 47 to 61, wherein the pH is adjusted with a base.

Embodiment 63. The process of the previous embodiment, wherein the base is a weak base.

Embodiment 64. The process of the previous embodiment, wherein the weak base is selected from sodium carbonate, potassium carbonate, caesium carbonate, ammonium hydroxide, methylamine, ethylamine, dimethylamine, diethylamine, triethylamine, diisopropylethylamine, sodium acetate, potassium acetate sodium formate, potassium formate, or mixtures thereof.

Embodiment 65. The process of the previous embodiment, wherein the weak base is selected from sodium carbonate, potassium carbonate, caesium carbonate, ammonium hydroxide, triethylamine, diisopropylethylamine, sodium acetate, potassium acetate, sodium formate, potassium formate, or mixtures thereof.

Embodiment 66. The hydromorphone monohydrate (I·H₂O) crystalline Form A of any of the embodiments 36 to 46 characterized by a PXRD comprising the following peaks: 11.2 and 15.1±0.2 degrees 2θ, referred to as Form A.

Embodiment 67. The hydromorphone monohydrate (I·H₂O) crystalline Form A of the previous embodiment characterized by a PXRD comprising the following peaks: 11.2, 15.1, and 25.4±0.2 degrees 2θ, referred to as Form A.

Embodiment 68. The hydromorphone monohydrate (I·H₂O) crystalline Form A of the previous embodiment characterized by a PXRD comprising the following peaks: 11.2, 15.1, 16.5, 25.4, and 25.7±0.2 degrees 2θ, referred to as Form A.

Embodiment 69. The hydromorphone monohydrate (I·H₂O) crystalline Form A of the previous embodiment characterized by a PXRD comprising the following peaks: 11.2, 12.3, 15.1, 16.5, 25.4, and 25.7±0.2 degrees 2θ, referred to as Form A.

Embodiment 70. The hydromorphone monohydrate (I·H₂O) crystalline Form A of the previous embodiment characterized by a PXRD comprising the following peaks: 11.2, 12.3, 15.1, 16.5, 23.2, 25.4, and ±0.2 degrees 2θ, referred to as Form A.

Embodiment 71. The hydromorphone monohydrate (I·H₂O) crystalline Form A of the previous embodiment characterized by a PXRD comprising the following peaks: 11.2, 12.3, 13.9, 15.1, 16.5, 23.2, 24.8, 25.4, 25.7, and 28.1±0.2 degrees 2θ, referred to as Form A.

Embodiment 72. The hydromorphone monohydrate (I·H₂O) crystalline Form A of the previous embodiment characterized by a PXRD comprising the following peaks: 11.2, 12.3, 13.9, 15.1, 16.5, 18.9, 19.3, 23.2, 24.8, 25.4, and 28.1±0.2 degrees 2θ, referred to as Form A.

Embodiment 73. The hydromorphone monohydrate (I·H₂O) crystalline Form A of the previous embodiment characterized by a PXRD comprising the following peaks: 11.2, 12.3, 13.9, 15.1, 16.5, 18.9, 19.3, 23.2, 23.6, 24.8, 25.4, 25.7, 28.1, 31.4, and 31.8±0.2 degrees 2θ, referred to as Form A.

Embodiment 74. The process of any of the embodiments 1 to 31, wherein the organic solvent comprises ethyl acetate, toluene, or mixtures thereof.

Embodiment 75. The process of the previous embodiment, wherein the organic solvent comprises ethyl acetate.

Embodiment 76. The process of embodiment 74, wherein the organic solvent comprises toluene.

Embodiment 77. The process of any of the embodiments 1 to 31 or 74 to 76, wherein the hydromorphone monohydrate (I·H₂O) is suspended or dissolved in step i. from 30 min to 48 h.

Embodiment 78. The process of the previous embodiment, wherein the hydromorphone monohydrate (I·H₂O) is suspended or dissolved in step i. from 1 h to 36 h.

Embodiment 79. The process of the previous embodiment, wherein the hydromorphone monohydrate (I·H₂O) is suspended or dissolved in step i. from 1.5 h to 24 h.

Embodiment 80. The process of the previous embodiment, wherein the hydromorphone monohydrate (I·H₂O) is suspended or dissolved in step i. from 1.5 h to 12 h.

Embodiment 81. The process of the previous embodiment, wherein the hydromorphone monohydrate (I·H₂O) is suspended or dissolved in step i. from 1.5 h to 6 h.

Embodiment 82. The process of the previous embodiment, wherein the hydromorphone monohydrate (I·H₂O) is suspended or dissolved in step i. from 1.5 h to 4 h.

Embodiment 83. The process of embodiment 79, wherein the hydromorphone monohydrate (I·H₂O) is suspended or dissolved in step i. from 4 h to 24 h.

Embodiment 84. The process of the previous embodiment, wherein the hydromorphone monohydrate (I·H₂O) is suspended or dissolved in step i. from 8 h to 24 h.

Embodiment 85. The process of the previous embodiment, wherein the hydromorphone monohydrate (I·H₂O) is suspended or dissolved in step i. from 12 h to 24 h.

Embodiment 86. The process of the previous embodiment, wherein the hydromorphone monohydrate (I·H₂O) is suspended or dissolved in step i. from 17 h to 22 h.

### Examples

Test 1. Powder X-Ray Diffraction Analysis (PXRD) Analysis are Performed as Follows:

Sample preparation: In order to acquire a powder diffraction pattern of the obtained solid, approximately 20 mg of the samples were prepared in a standard sample holder using two foils of polyacetate.

Data acquisition: Powder diffraction patterns were acquired on a Bruker D8 Advance Series 2Theta/Theta powder diffraction system using CuKα1-radiation (1.54060 Å) in transmission geometry. The system is equipped with a V\check{A}NTEC-1 single photon counting PSD, a Germanium monochromator, a ninety positions auto changer sample stage, fixed divergence slits and radial soller.

Programs used: Data collection with DIFFRAC plus XRD Commander V.2.5.1 and evaluation with High Score Plus 4.9 (Malvern Panalytical).

Measurement conditions: The samples were measured at room temperature in a range from 4° to 40° in degrees 2θ in a 1 hour measurement, using an angular step of 0.033° and a time per step of 2930.45 s.

### Example 1. Preparation of Hydromorphone Monohydrate (I·H₂O) from Hydromorphone Hydrochloride (I·HCl)

1.002 g (3.11 mmol) of I·HCl was dissolved in 10 mL of water under nitrogen stream and cooled to 0/10° C. 0.278 g (2.62 mmol) of sodium carbonate was portionwise added under stirring to adjust pH to 9.0. The mixture was stirred for 3 h at 0/10° C. The resulting suspension was filtered, the solid was washed with cold water and dried at 50° C. under vacuum (55 mbar). 0.774 g (82% yield) of I·H₂O was obtained.

FTIR (FIG. 1): 3547, 2925, 1721, 1377, 973, 749 cm⁻¹.

DSC endotherm at 112° C. & 276° C., loss of water and I endotherm respectively, as shown in FIG. 2.

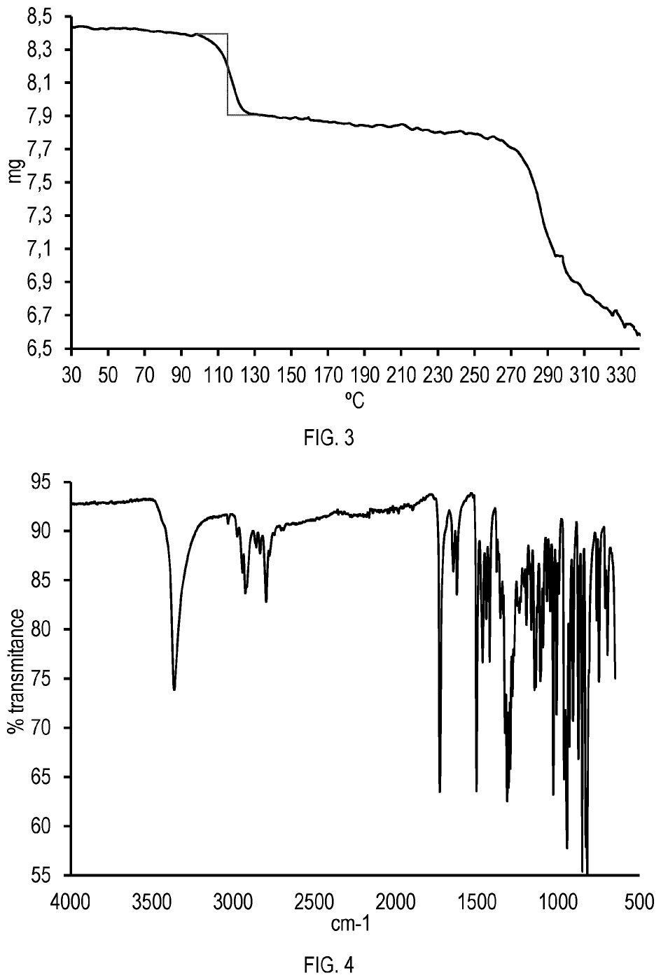

TGA 5.8%, loss of water between 98° C. and 115° C., as shown in FIG. 3.

MW I·H₂O: 303.36.

MW H₂O: 18.02.

I·H₂O has a 5.94% of water.

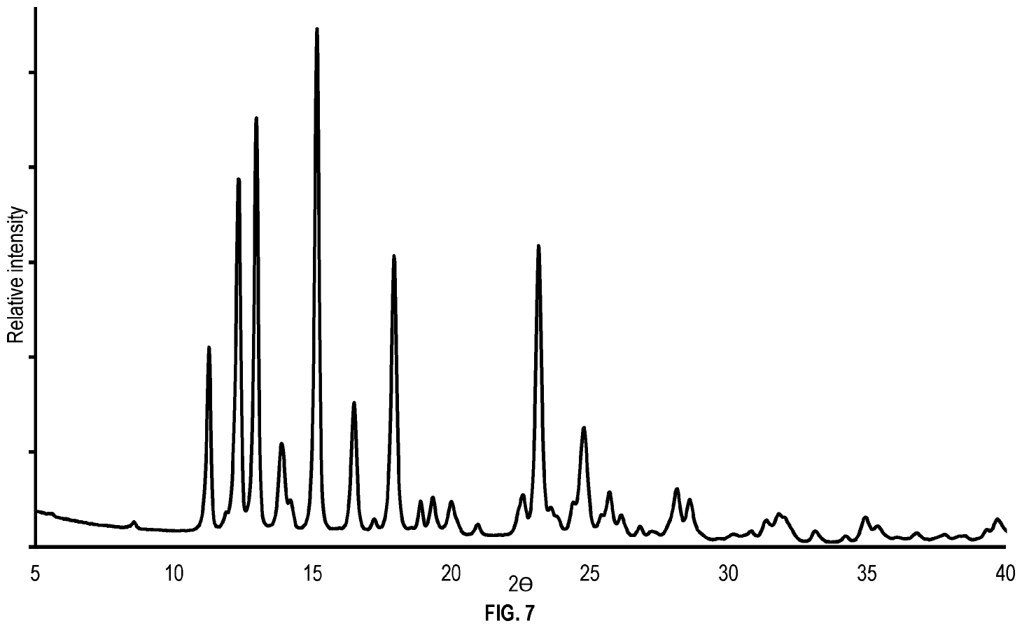

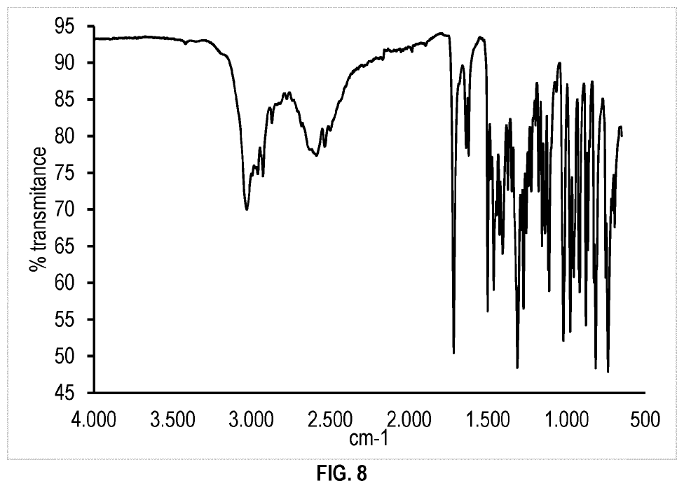

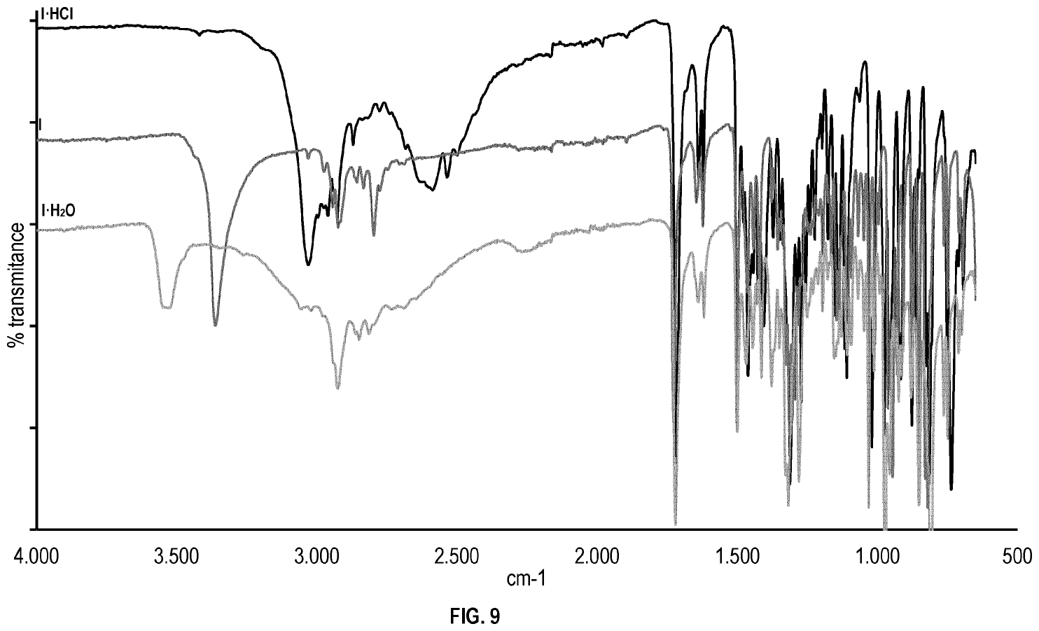

PXRD Form A, as shown in FIG. 7 with the following peaks:

| Angle (2θ) | Rel. Intensity (%) | d Value (Å) |
|---|---|---|
| 5.6 | 0.4 | 15.87 |
| 8.5 | 1.3 | 10.35 |
| 11.2 | 36.6 | 7.86 |
| 11.9 | 2.6 | 7.45 |
| 12.3 | 70.7 | 7.18 |
| 13.0 | 81.9 | 6.83 |
| 13.9 | 17.9 | 6.37 |
| 14.2 | 5.3 | 6.23 |
| 15.1 | 100 | 5.84 |
| 16.5 | 26.7 | 5.37 |
| 17.2 | 2.2 | 5.15 |
| 17.9 | 56.1 | 4.94 |
| 18.7 | 0.6 | 4.75 |
| 18.9 | 6 | 4.69 |
| 19.3 | 7.6 | 4.59 |
| 20.0 | 6.6 | 4.44 |
| 20.2 | 1.3 | 4.39 |
| 21.0 | 2.2 | 4.24 |
| 21.7 | 0.1 | 4.09 |
| 22.5 | 7 | 3.94 |
| 23.2 | 58.9 | 3.84 |
| 23.6 | 3.6 | 3.77 |
| 23.8 | 2.6 | 3.73 |
| 24.4 | 5.3 | 3.65 |
| 24.8 | 22.1 | 3.59 |
| 25.4 | 3.2 | 3.5 |
| 25.7 | 8.7 | 3.46 |
| 26.1 | 4.2 | 3.41 |
| 26.8 | 2.2 | 3.32 |
| 27.2 | 1.1 | 3.27 |
| 28.1 | 10 | 3.17 |
| 28.6 | 7.2 | 3.12 |
| 29.7 | 0.5 | 3.01 |
| 30.2 | 1.3 | 2.96 |
| 30.8 | 1.9 | 2.9 |
| 31.4 | 4.2 | 2.85 |
| 31.8 | 4.9 | 2.81 |
| 32.1 | 3.3 | 2.79 |
| 33.1 | 2.3 | 2.7 |
| 34.2 | 1.2 | 2.62 |
| 34.9 | 4.6 | 2.57 |
| 35.4 | 3 | 2.53 |
| 36.1 | 0.6 | 2.49 |
| 36.8 | 1.4 | 2.44 |
| 37.5 | 0.3 | 2.4 |
| 37.8 | 1.1 | 2.38 |
| 38.3 | 0.7 | 2.35 |
| 38.5 | 0.6 | 2.33 |
| 39.3 | 1.2 | 2.29 |
| 39.7 | 3.8 | 2.27 |

### Example 2: Preparation of Hydromorphone Base (I) from Hydromorphone Monohydrate (I·H₂O) in Ethyl Acetate

0.306 g of I·H₂O (1.00 mmol) was suspended in 1.5 mL of anhydrous ethyl acetate at 20/25° C. under nitrogen stream for 2 h. After cooling to 0/10° C. for 1 h, the resulting suspension was filtered and the solid washed twice with 1 mL of cold ethyl acetate. The solid was dried at 50° C. under vacuum to obtain 0.257 g (89% yield) of I.

FTIR (FIG. 4): 3361, 2924, 2797, 1727, 1502, 1314, 946 cm⁻¹.

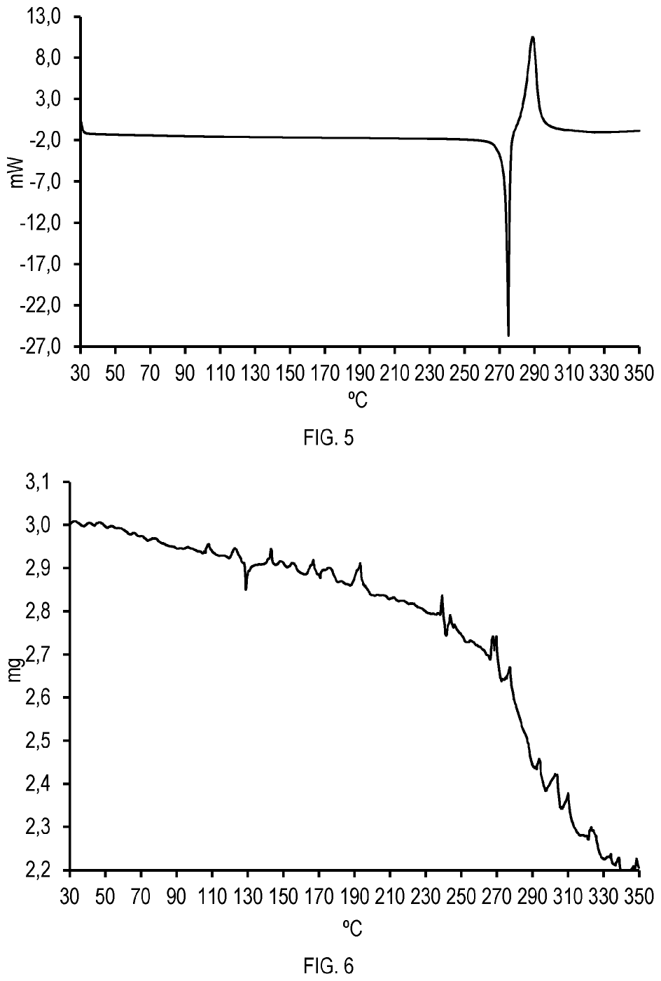

DSC at 275° C., as shown in FIG. 5.

### Example 3: Preparation of Hydromorphone Base (I) from Hydromorphone Monohydrate (I·H₂O) in a Battery of Solvents

0.100 g of hydromorphone monohydrate (I·H₂O) (0.33 mmol) was suspended in 0.5 mL of the anhydrous solvent listed in the table below at 20/25° C. under nitrogen stream for 2 h. After cooling to 0/10° C. for 1 h, the resulting suspension was filtered, and the solid was washed four times with 0.5 mL of the respective cold solvent. The solid was dried at 50° C. under vacuum. Results are reported in the following table:

| solvent | yield | hydromorphone purity |
|---|---|---|
| Isopropyl acetate | 69% | Mixture of monohydrate and base form |
| Methyl tert-butyl ether | 67% | Mixture of monohydrate and base form |
| Tetrahydrofuran | 48% | base form |
| Methanol | 40% | base form |
| Heptane | 72% | Mixture of monohydrate and base form |
| Toluene | 71% | Mixture of monohydrate and base form |
| Methyl isobutyl ketone | 64% | base form with traces of monohydrate form |

### Example 4. Preparation of Hydromorphone Monohydrate (I·H₂O) from Hydromorphone Hydrochloride (I·HCl)

2.0 g (6.23 mmol) of I·HCl were dissolved in 20 mL of water under nitrogen stream and cooled to 0/10° C. Sodium carbonate (0.766 g, 7.22 mmol) was portionwise added at 0/10° C. under stirring to adjust pH to 9.0. The mixture was stirred for 2.5 h at 0/10° C. The resulting suspension was filtered and the solid was washed several times with cold water to completely remove NaCl. The solid was dried at 50° C. under vacuum (55 mbars) to obtain 1.754 g (92.8% yield) of I·H₂O. The FTIR matches with the monohydrate obtained in Example 1.

### Example 5: Preparation of Hydromorphone Base (I) from Hydromorphone Monohydrate (I·H₂O) in Toluene

0.898 g of I·H₂O (2.96 mmol) were suspended in 4.5 mL of toluene at 30/35° C. under nitrogen stream for 19 h. After cooling to 0/10° C. for 1 h, the resulting suspension was filtered and the solid washed twice with 2 mL of toluene. The solid was dried at 50° C. under vacuum to obtain 0.788 g (88% yield) of I. The FTIR matches with the hydromorphone base (I) obtained in Example 2.

### REFERENCES

CSUK, R., et al., Towards an Efficient Preparation of Hydromorphone. Synthesis 2012, Vol. 44, N° 18, pages 2840-2842. DOI: 10.1055/s-0031-1291151.
ERBING, E. et al., General, Simple, and Chemoselective Catalysts for the Isomerization of Allylic Alcohols:
The Importance of the Halide Ligand. Chem. Eur. J. 2016, Vol. 22, N° 44, pages 15659-15663. DOI:
FISCHER, R., et al., Zum Nachweis narkotisch wirkender Substanzen in biologischem Material. Mikrochim Acta 1949, Vol. 34, N° 3, pages 257-268. DOI: 10.1007/BF01412693.
GOMEZ, AB. et al. Transition metal-catalyzed redox isomerization of codeine and morphine in water. RSA Adv. 2014, Vol. 4, N° 20, pages 39519-39522. DOI: 10.1039/C₄RA07735K.
MAZUREK, J., et al., Two orthorhombic polymorphs of hydromorphone. Acta Cryst 2016, Vol. 72, N° 5, pages 730-733. DOI: 10.1107/S2056989016006563.
MERK INDEX, 2006, O'Neil, M. J. (ed.). The Merck Index—An Encyclopedia of Chemicals, Drugs, and Biologicals. Whitehouse Station, NJ: Merck and Co., Inc., 2006., p. 121.
MURPHY, B., et al., Conversion of Thebaine to Oripavine and Other Useful Intermediates for the Semisynthesis of Opiate-Derived Agents: Synthesis of Hydromorphone. Adv. Synth. Catal. 2014, Vol. 256, N° 11-12, pages 2679-2687. DOI: 10.1002/adsc.201400445.
RAPOPORT, H., et al., The preparation of some dihydro ketones in the morphine series by Oppenauer oxidation. J. Org. Chem. 1950, Vol. 15, N° 5, pages 1103-1107. DOI: 10.1021/jo01151a029.
WANG, M. et al., The Intriguing Effects of Substituents in the N-Phenethyl Moiety of Norhydromorphone: A Bifunctional Opioid from a Set of “Tail Wags Dog” Experiments. Molecules 2020, Vol 25, N° 11, 2640.; DOI:

1. A process for preparing hydromorphone base (I) comprising:
i. suspending or dissolving hydromorphone monohydrate (I·H₂O) in at least an organic solvent, and
ii. isolating hydromorphone base (I).

2. The process of claim 1, wherein the organic solvent comprises methanol, ethanol, 1-propanol, 2-propanol, 1-butanol, 2-butanol, isobutanol, tert-butanol, methyl acetate, ethyl acetate, isopropyl acetate, tetrahydrofuran, dichloromethane, acetone, methyl isobutyl ketone, acetonitrile, propionitrile, butyronitrile, diethyl ether, 1,4-dioxane, methyl tert-butyl ether, pentane, cyclopentane, hexane, cyclohexane, heptane, toluene, chloroform, or mixtures thereof.

3. The process of claim 1, wherein the organic solvent comprises a polar organic solvent.

4. The process of claim 3, wherein the polar organic solvent is ethyl acetate.

5. The process of claim 1, wherein the organic solvent comprises a non-polar organic solvent.

6. The process of claim 5, wherein the non-polar organic solvent is toluene.

7. The process of claim 1, wherein the organic solvent is an anhydrous organic solvent.

8. The process of claim 1, wherein the hydromorphone monohydrate (I·H₂O) is suspended or dissolved in step i. at 15-80° C.

9. The process of claim 8, wherein the hydromorphone monohydrate (I·H₂O) is suspended or dissolved in step i. at 20-25° C.

10. The process of claim 9, wherein the hydromorphone monohydrate (I·H₂O) is suspended or dissolved in step i. at 25-35° C.

11. The process of claim 1, wherein isolating step ii. is performed between −5 and 25° C.

12. The process of claim 11, wherein isolating step ii. is performed between 0 and 10° C.

13. Hydromorphone monohydrate (I·H₂O),

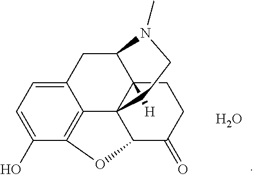

14. The hydromorphone monohydrate (I·H₂O) of claim 13 in a crystalline form comprising a DSC with two endotherms peaks, one at 112±4° C. and a second at 276±2° C., referred to as Form A.

15. The hydromorphone monohydrate (I·H₂O) crystalline form of claim 13, comprising an FTIR comprising the following peaks: 3547, 2925, 1721, 1377, 973, 749±5 cm⁻¹, referred to as Form A.

16. The hydromorphone monohydrate (I·H₂O) crystalline form of claim 13, comprising characterized by an PXRD comprising the following peaks: 11.2 and 15.1±0.2 degrees 2θ, referred to as Form A.

17. The hydromorphone monohydrate (I·H₂O) crystalline form of claim 16, comprising an PXRD comprising the following peaks: 11.2, 15.1, and 25.4, ±0.2 degrees 2θ, referred to as Form A.

18. The hydromorphone monohydrate (I·H₂O) crystalline form of claim 17, comprising characterized by an PXRD comprising the following peaks: 11.2, 12.3, 13.9, 15.1, 16.5, 18.9, 19.3, 23.2, 23.6, 24.8, 25.4, 25.7, 28.1, 31.4, and 31.8±0.2 degrees 2θ, referred to as Form A.

19. A process for preparing hydromorphone monohydrate (I·H₂O) comprising
a) dissolving a hydromorphone salt in a solvent medium comprising water,
b) adjusting the pH of the mixture of step a) between 8 and 10, and
c) isolating hydromorphone monohydrate (I·H₂O) from the mixture of step b);
wherein:
step c) is performed between −5 and 25° C.

20. The process of claim 19, wherein the solvent medium comprising water comprises at least 50% water.

21. The process of claim 20, wherein the solvent medium comprising water comprises at least 95% water.

22. The process of claim 19, wherein the pH is adjusted between 8.2 and 9.8.

23. The process of claim 22, wherein the pH is adjusted between 8.8 and 9.2.

24. The process of claim 19, wherein in step b) the pH is adjusted with a weak base.

25. The process of claim 24, wherein the weak base is sodium carbonate, potassium carbonate, caesium carbonate, ammonium hydroxide, methylamine, ethylamine, dimethylamine, diethylamine, triethylamine, diisopropylethylamine, sodium acetate, potassium acetate sodium formate, potassium formate, or mixtures thereof.

26. The process of claim 25, wherein the weak base is sodium carbonate.

27. The process of claim 19 wherein hydromorphone salt is hydromorphone hydrochloride (I·HCl).

28. The process of claim 19, wherein step c) is performed between 0 and 10° C.

29. The process according to claim 1, wherein the hydromorphone monohydrate (I·H₂O) is obtained by
a) dissolving a hydromorphone salt in a solvent medium comprising water,
b) adjusting the pH of the mixture of step a) between 8 and 10, and
c)isolating hydromorphone monohydrate (I·H₂O) from the mixture of step b),
wherein:
step c) is performed between −5 and 25° C.

30. The process according to claim 1, wherein the hydromorphone monohydrate (I·H₂O)

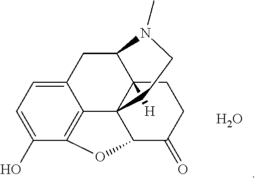

31. The hydromorphone monohydrate (I·H₂O) of claim 13 for use in therapy.

32. The hydromorphone monohydrate (I·H₂O) for use in claim 31, wherein the therapy is pain management.
